(19) United States
(12) Patent Application Publication
(10) Pub. No.: US 2019/0000693 A1
(43) Pub. Date: Jan. 3, 2019

(54) ABSORBENT ARTICLE AND METHOD FOR MANUFACTURING ABSORBENT ARTICLE

(71) Applicant: UNICHARM CORPORATION, Shikokuchuo-shi, Ehime (JP)

(72) Inventors: Asami SHIMA, Kanonji-shi, Kagawa (JP); Toshiyuki TANIO, Kanonji-shi, Kagawa (JP)

(73) Assignee: Unicharm Corporation, Shikokuchuo-shi, Ehime (JP)

(21) Appl. No.: 16/065,164

(22) PCT Filed: Sep. 27, 2016
(86) PCT No.: PCT/JP2016/078471

§ 371 Date: Jun. 22, 2018

(30) Foreign Application Priority Data
Dec. 28, 2015 (JP) 2015-256289

(51) Int. Cl. A61F 13/535; A61F 13/476; A61F 13/56; A61F 13/15

(52) U.S. Cl. CPC A61F 13/535; A61F 13/476; A61F 2013/51452; A61F 13/15707; A61F 13/5616

(57) ABSTRACT

An absorbent article having a longitudinal direction, a width direction, and a thickness direction that intersect with one another, the absorbent article includes: an absorbent body that absorbs liquid; a back sheet disposed on a wearer's non-skin side of the absorbent body, the back sheet made of a nonwoven fabric; and an anti-displacement adhesive portion formed with an adhesive, the adhesive provided on a non-skin side of the back sheet, the back sheet including a compressed region that is compressed in the thickness direction, the absorbent article including a portion where the anti-displacement adhesive portion overlaps the compressed region.

### TECHNICAL FIELD

The present disclosure relates to an absorbent article and a method for manufacturing an absorbent article.

### BACKGROUND ART

Absorbent articles that absorb excreted fluid such as menstrual blood are known (e.g., sanitary napkins).

Such an absorbent article has a longitudinal direction, a width direction, and a thickness direction that are orthogonal to one another. Further, the absorbent article includes an absorbent body, mainly made of liquid absorbent fibers, between a top sheet and a back sheet. In the absorbent article, such a technique is disclosed that the back sheet provided on the non-skin side of the absorbent body is formed with a nonwoven fabric, to enhance breathability and flexibility (for example, PTL 1).

### CITATION LIST

### Patent Literature

[PTL 1] Japanese Patent No. 3851737

### SUMMARY OF INVENTION

### Technical Problem

In general, an anti-displacement adhesive portion for fixing the absorbent article to a wearer's underwear when worn is provided on the non-skin side (i.e., back sheet) of the absorbent article. In an absorbent article of PTL 1, the back sheet is formed with a nonwoven fabric, and the surface thereof is in a fluffy state. When such a surface of the nonwoven fabric is provided with an anti-displacement adhesive portion, a joining force between an adhesive forming the anti-displacement adhesive portion and the nonwoven fabric surface is weak. As a result, when removing the used absorbent article from underwear, the adhesive is easily peeled off from the nonwoven fabric (back sheet), so that the adhesive may remain on the underwear.

The present disclosure has been made in view of such an issue, and an object thereof is that, in an absorbent article including a back sheet made of a nonwoven fabric, an adhesive forming an anti-displacement portion is suppressed from being peeled off from the back sheet.

### Solution to Problem

A primary aspect of the present disclosure is an absorbent article having a longitudinal direction, a width direction, and a thickness direction that intersect with one another, the absorbent article comprising: an absorbent body that absorbs liquid; a back sheet disposed on a wearer's non-skin side of the absorbent body, the back sheet made of a nonwoven fabric; and an anti-displacement adhesive portion formed with an adhesive, the adhesive provided on a non-skin side of the back sheet, the back sheet including a compressed region that is compressed in the thickness direction, the absorbent article including a portion where the anti-displacement adhesive portion overlaps the compressed region.

Other features of the present disclosure will become clear from the descriptions of the present specification with reference to the accompanying drawings.

### Advantageous Effects of Invention

According to the present disclosure, it is possible that, in an absorbent article including a back sheet made of a nonwoven fabric, an adhesive forming an anti-displacement portion is suppressed from being peeled off from the back sheet.

### BRIEF DESCRIPTION OF THE DRAWINGS

FIG. 1 is a schematic plan view illustrating a napkin 1 when viewed from a skin side in a thickness direction.

FIG. 2 is a schematic plan view illustrating a napkin 1 when viewed from a non-skin side in a thickness direction.

FIG. 3 is a schematic sectional view taken along a line A-A of FIG. 1.

FIGS. 4A and 4B are diagrams illustrating states of a surface of a nonwoven fabric before and after being subjected to a compression process.

FIGS. 5A and 5B are diagrams illustrating a wettability test method.

FIG. 6 is a table representing results of a wettability test.

FIG. 7 is a table representing results of a surface test.

FIG. 8 is a plan view illustrating a relationship between an anti-displacement adhesive portion 130 and a compressed region 200.

FIG. 9 is a plan view illustrating a relationship between a main-body compressed region 201, and a leak-proof sheet 40 and a leak-proof sheet adhesive portion 140.

FIG. 10 is a table representing results of a tensile test.

FIG. 11 is a diagram illustrating a modified example of a main-body compressed region 201.

FIG. 12 is a diagram illustrating a case where high density portions 201h and low-density portions 201l are continuously arranged in a longitudinal direction in a compressed region 200.

### DESCRIPTION OF EMBODIMENTS

At least following matter will become clear from the descriptions of the present specification with reference to the accompanying drawings.

An absorbent article having a longitudinal direction, a width direction, and a thickness direction that intersect with one another, the absorbent article comprising: an absorbent body that absorbs liquid; a back sheet disposed on a wearer's non-skin side of the absorbent body, the back sheet made of a nonwoven fabric; and an anti-displacement adhesive portion formed with an adhesive, the adhesive provided on a non-skin side of the back sheet, the back sheet including a compressed region that is compressed in the thickness direction, the absorbent article including a portion where the anti-displacement adhesive portion overlaps the compressed region.

According to such an absorbent article, the adhesion of the adhesive forming the anti-displacement adhesive portion is strengthened in the compressed region in which the surface in the back sheet is formed smooth. Thus, the adhesive is less likely to be peeled off in the region in which the compressed region overlaps (lies on) the anti-displacement adhesive portion. Accordingly, when removing the used absorbent article from underwear, it is possible to restrain the adhesive forming the anti-displacement adhesive portion from being peeled and remaining on underwear.

In such an absorbent article, it is preferable that the back sheet includes an uncompressed region in a region other than a region where the compressed region is formed, and the uncompressed region overlaps a region where the anti-displacement adhesive portion is not disposed, in the back sheet.

According to such an absorbent article, the uncompressed region, where the surface of the nonwoven fabric is maintained softer than that in the compressed region, is included on the back sheet, so that preferable texture is ensured on the non-skin side of the absorbent article. As a result, it is possible to restrain the texture from deteriorating even when the back sheet contacts the skin of a wearer while wearing the absorbent article.

In such an absorbent article, it is preferable that the compressed region includes high density portions having a high density and low density portions having a density lower than the density of the high density portions.

According to such an absorbent article, in the low-density portions, the nonwoven fabric surface is likely to be maintained softer than that in the high density portions. Thus, it is possible to maintain softness and texture to some extent in the compressed region and the anti-displacement adhesive portion overlapping the compressed region.

In such an absorbent article, it is preferable that the compressed region includes the high density portions disposed intermittently in the longitudinal direction, the high density portions having a predetermined length in the width direction, and the low density portions each disposed between every two, of the high density portions, adjacent to each other in the longitudinal direction, the low density portions having a predetermined length in the width direction.

According to such an absorbent article, in removing the used absorbent article, even if the adhesive is peeled in a low-density portion when the absorbent article is pulled along the longitudinal direction, such peeling of the adhesive is likely to be restrained from being continuously propagated in the longitudinal direction due to the high density portion adjacent to the low-density portion in the longitudinal direction. That is, this makes it easier to restrain the adhesive from being peeled continuously along the longitudinal direction.

In such an absorbent article, it is preferable that the high density portions each having a length, in the longitudinal direction, longer than a length in the longitudinal direction of each of the low density portions.

According to such an absorbent article, the rate of the area of the high density portions in the compressed region is larger than the rate of the area of the low-density portion. Thus, a smooth region is larger in the back sheet, so that the adhesion of the adhesive can be strengthened.

In such an absorbent article, it is preferable that a contact angle of water in the compressed region is equal to or greater than 100 degrees and equal to or smaller than 120 degrees.

According to such an absorbent article, since the wettability is higher in the compressed region of the back sheet and the surface thereof is smooth, the adhesion of the adhesive can be sufficiently strengthened. Accordingly, when the contact angle of water in the compressed region is in a range of being equal to or greater than 100 degrees and equal to or smaller than 120 degrees, the adhesive can be less likely to be peeled.

In such an absorbent article, it is preferable that the back sheet includes an uncompressed region in a region other than a region where the compressed region is formed, and the absorbent article includes wing portions extending outward from both side portions in the width direction of the absorbent body, and the anti-displacement adhesive portion includes a main-body anti-displacement portion formed along the longitudinal direction in a central portion in the width direction, and a wing anti-displacement portion formed on a non-skin side of the wing portion, and the uncompressed region is provided in a region between the main-body anti-displacement portion and the wing anti-displacement portion in the width direction.

According to such an absorbent article, the reference folding lines, which serve as references along which the wing portions are folded when the absorbent article is worn, are formed soft. Thus, the wing portions are easily folded. Further, the reference folding lines are portions more likely to directly contact both legs of a wearer while wearing the absorbent article. Thus, if such portions are formed into uncompressed regions, the texture thereof is soft, which is less likely to give uncomfortableness to the wearer.

In such an absorbent article, it is preferable that the back sheet includes an uncompressed region in a region other than a region where the compressed region is formed, and both ends in the width direction of the absorbent body overlap the uncompressed region.

According to such an absorbent article, both edges in the width direction of the absorbent article, which are to be caught by the inguinal regions of a wearer when wearing the absorbent article overlap the soft uncompressed regions. Thus, the feel in the inguinal regions of the wearer can be made preferable.

In such an absorbent article, it is preferable that the compressed region does not overlap an outer peripheral edge of the absorbent article.

According to such an absorbent article, flexibility can be ensured by forming the outer peripheral edge (edge) into the uncompressed region. Accordingly, it is possible to restrain the edge from being caught by a wearer's body and restrain the wearer from feeling uncomfortable when the absorbent article is worn.

In such an absorbent article, it is preferable to further comprise a top sheet disposed on a wearer's skin side of the absorbent body and a surround seal portion joining the top sheet and the back sheet at an outer peripheral portion of the absorbent article, wherein the compressed region does not overlap the surround seal portion.

According to such an absorbent article, it is possible that a wearer is less likely to feel hardness of the surround seal portion when the absorbent article is worn, resulting in being less likely to feel awkwardness.

Further a method for manufacturing an absorbent article will be made clear which comprises: forming a compressed region by performing a compression process, in the thickness direction, onto a predetermined region of a back sheet made of a nonwoven fabric; forming an anti-displacement adhesive portion including a portion overlapping the compressed region by providing an adhesive on a non-skin side of the back sheet; and joining an absorbent body that absorbs liquid to a skin side of the back sheet.

According to such a method for manufacturing an absorbent article, it is possible to strengthen the adhesion of the adhesive forming the anti-displacement adhesive portion in the compressed region where the surface of the back sheet is formed smooth. Thus, the adhesive is made less likely to be peeled in a region where the compressed region overlaps the anti-displacement adhesive portion. Accordingly, when removing the used absorbent article from underwear, it is possible to manufacture an absorbent article capable of restraining the adhesive forming the anti-displacement adhesive portion from being peeled off and remaining on the underwear.

### Embodiments

### Basic Configuration of Sanitary Napkin

A sanitary napkin 1 (hereinafter, also simply referred to as the napkin 1) will be described as one example of an absorbent article according to an embodiment of the present disclosure. Note that, in the following description, although the sanitary napkin will be described as an example of the absorbent article, the absorbent article according to an embodiment of the present disclosure includes the so-called vaginal discharge sheet (e.g., panty liner) and the like, and is not limited to the sanitary napkin.

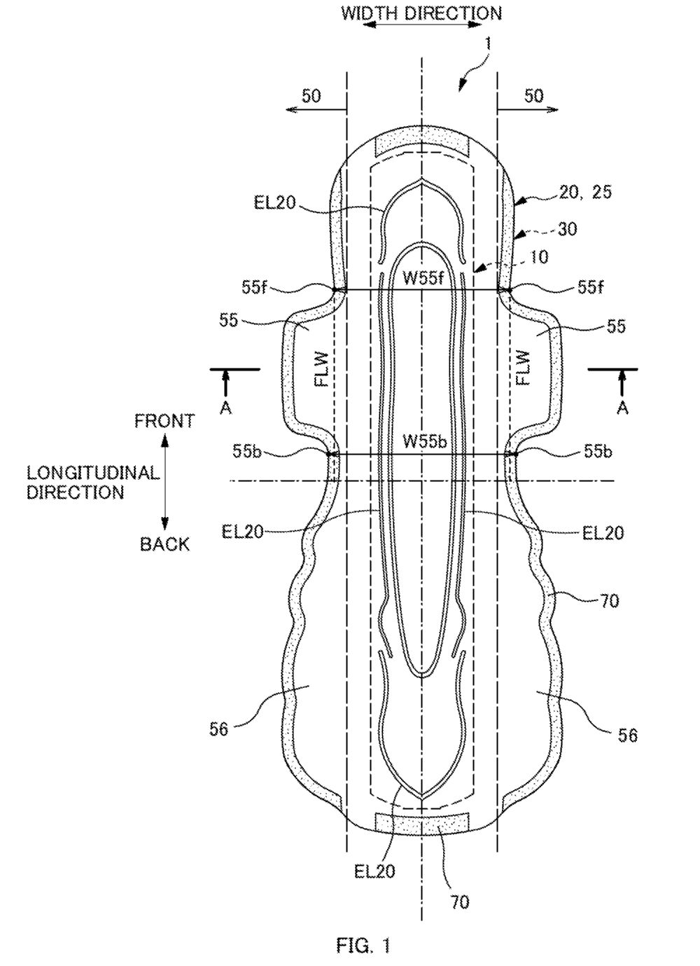

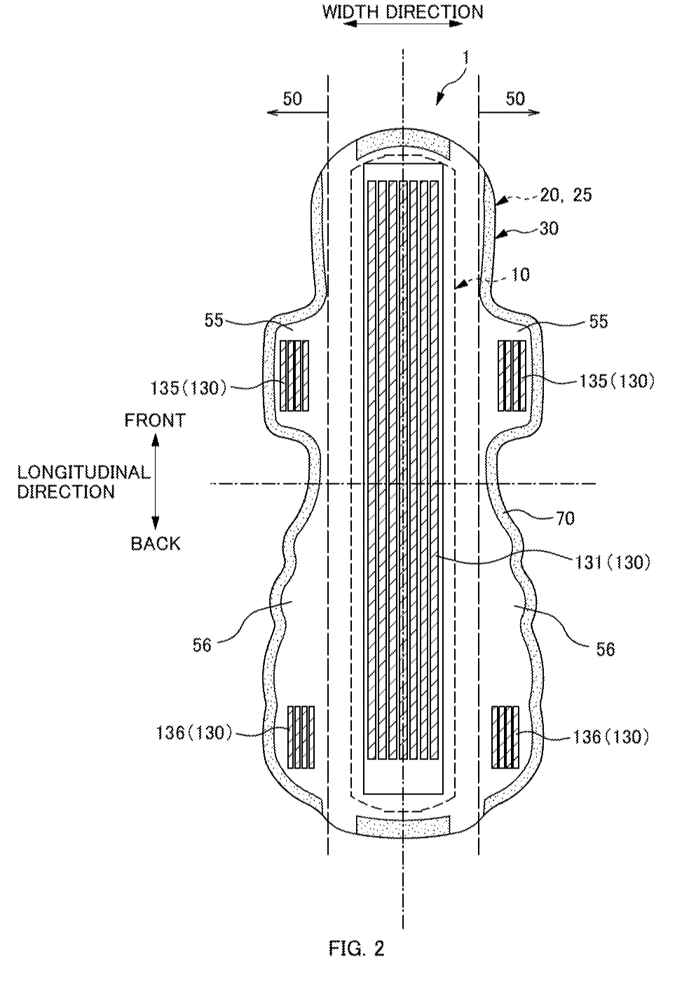

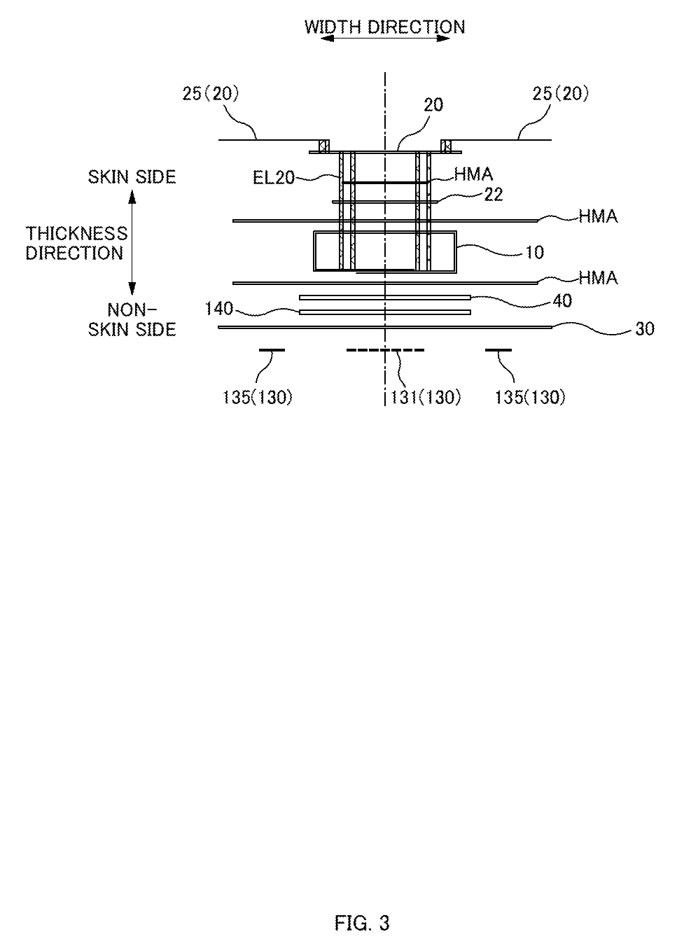

FIG. 1 is a schematic plan view illustrating the napkin 1 when viewed from the skin side in a thickness direction. FIG. 2 is a schematic plan view illustrating the napkin 1 when viewed from the non-skin side in the thickness direction. FIG. 3 is a schematic sectional view taken along a line A-A of FIG. 1. In the following description, directions are defined as illustrated in FIGS. 1 to 3. That is, defined are a “longitudinal direction” along the longitudinal direction of the product which is the napkin 1, a “width direction” that is orthogonal to the longitudinal direction and along a lateral direction of the production which is the napkin 1, and the “thickness direction” orthogonal to the longitudinal direction and the width direction. The longitudinal direction includes a “front side” which is to be the front side of a wearer when wearing the napkin 1, and a “back side” which is to be the back side of the wearer. The thickness direction includes a “skin side” which is the side (upper side in FIG. 3) to contact the skin of the wearer when wearing the napkin 1, and a “non-skin side” which is the opposite side (lower side in FIG. 3).

The napkin 1 includes: an absorbent body 10 that absorbs liquid; a top sheet 20 disposed on the wearer's skin side of the absorbent body 10; a back sheet 30 disposed on the wearer's non-skin side of the absorbent body 10; a leak-proof sheet 40 disposed between the absorbent body 10 and the back sheet 30; and side flaps 50 provided outside in the width direction of the absorbent body 10. These members are joined to each other such that each of the members is joined to another member adjacent in the thickness direction, using an adhesive such as a hot-melt adhesive HMA (see FIG. 3).

The absorbent body 10 is a longitudinal member that is elongated in the longitudinal direction, contains hydrophilic fibers, pulp, and the like, and is capable of efficiently absorbing/holding liquids such as bodily fluids and blood discharge. The absorbent body 10 is formed, for example, such that hydrophilic fibers or powders are deposited by the air laying method.

The top sheet 20 is a liquid permeable sheet member that allows liquid such as bodily fluids to pass therethrough, and formed of, for example, a nonwoven fabric. However, a member other than the nonwoven fabric may be used as long as the member is a sheet member allowing liquid to pass therethrough as well as the member is safe when contacts the skin of a wearer when wearing the napkin 1. For example, the member may be a woven fabric or a mesh sheet. The top sheet 20 is larger, in the longitudinal direction and the width direction, than the absorbent body 10, and is capable of covering the entire region of the absorbent body 10. Further, a second sheet 22 constituted by a member equivalent to that of the top sheet 20 is provided between the top sheet 20 and the absorbent body 10 in the thickness direction. However, the second sheet 22 is not necessarily provided.

Further, side sheets 25 extending outward in the width direction are joined to the top sheet 20 on both sides in the width direction, respectively, and these side sheets 25 formed the side flaps 50, respectively, which will be described later. Note that the side flap 50 may be formed by partially extending the top sheet 20 outside the absorbent body 10 in the width direction, without using the side sheet 25 separate from the top sheet 20. Accordingly, in the present description, the side sheets 25 are considered as parts of the top sheet 20.

The back sheet 30 is a sheet member made of a nonwoven fabric, and is disposed on the side farthest from the skin of a wearer, in the napkin 1 (see FIG. 3). In many cases, a back sheet of a conventional napkin is formed of a liquid-impermeable film or the like. However, in the napkin 1 according to an embodiment of the present disclosure, since the back sheet 30 is formed of a nonwoven fabric, breathability and flexibility are enhanced as compared with the conventional napkin. In an embodiment of the present disclosure, the back sheet 30 is formed of, for example, an SMS (Spunbond/Melt-blown/Spunbond) nonwoven fabric. SMS nonwoven fabric includes a melt-blown layer having a number of fabrics larger than the numbers of fabrics in a spunbond layer and other dry-type nonwoven fabrics when compared with the same basis weight. Thus, its texture is stable, and even if its basis weight is low, the area without fibers is smaller, so that the adhesive area is increased. That is, SMS nonwoven fabric has a large number of fibers contained per unit volume, and thus the effects of a compression process, which will be described later, are likely to be exerted more remarkably, as compared with other nonwoven fabrics, such as an air-through nonwoven fabric. Thus, SMS nonwoven fabric is suitable for an embodiment of the present disclosure. Parts of the back sheet 30 extend outside the absorbent body 10 in the width direction, to form the side flaps 50, which will be described later together with the side sheets 25, respectively.

The compression process in the thickness direction is performed in a region in the back sheet 30, to form a compressed region 200. Further, in a predetermined region on the non-skin side of the back sheet 30, an adhesive such as a hot-melt adhesive (HMA) is provided to form an anti-displacement adhesive portion 130. As illustrated in FIG. 2, the anti-displacement adhesive portion 130 includes a belt-shaped main-body anti-displacement portion 131 foamed along the longitudinal direction in a central portion in the width direction of the back sheet 30, wing anti-displacement portions 135 formed in the side flaps 50, and hip-flap anti-displacement portions 136. Such an anti-displacement adhesive portion 130 is attached to underwear of a wearer when wearing the napkin 1, so that the position of the napkin 1 will not be displaced. Note that when the napkin 1 is not used, the anti-displacement adhesive portion 130 is covered with a protection sheet (not shown). In a state of being covered with such a protection sheet, adhesion of the anti-displacement adhesive portion 130 does not appear. The compressed region 200 and the anti-displacement adhesive portion 130 will be described later in detail.

As illustrated in FIG. 3, the leak-proof sheet 40 is an impermeable sheet member joined to the non-skin-side surface of the absorbent body 10 and the skin-side surface of the back sheet 30, each using an adhesive such as a hot-melt adhesive (HMA). In an embodiment of the present disclosure, the leak-proof sheet 40 is formed of a film that is mainly made of, for example, polyethylene and propylene, a breathable resin film, or the like. Since the leak-proof sheet 40 is impermeable, the liquid absorbed by the absorbent body 10 through the top sheet 20 is suppressed from seeping on the clothes side (non-skin side) such as underwear while the napkin 1 is worn.

In the napkin 1, the back sheet 30 and the leak-proof sheet 40 are joined via a leak-proof sheet adhesive portion 140 that is formed by an adhesive provided on the skin side of the back sheet 30 (see FIG. 3). The arrangement of the leak-proof sheet adhesive portion 140 and the like will be described later in detail.

The side flaps 50 are formed such that the back sheet 30 and the side sheets 25 (top sheet 20) extending outward from both sides in the width direction of the absorbent body 10 are laminated in the thickness direction and joined (see FIGS. 1 and 2).

The side flaps 50 according to an embodiment of the present disclosure respectively include, as illustrated in FIG. 1, wing portions 55 extending outward in the width direction from a region on the front side with respect to the center in the longitudinal direction of the absorbent body 10, and hip flap portions 56 extending outward in the width direction from a region on the back side in the longitudinal direction with respect to the wing portions 55.

When the napkin 1 is worn, the wing portions 55 are folded toward the non-skin side, to attach the wing anti-displacement portion 135 to a crotch portion of the underwear of a wearer, so that the napkin 1 is less likely to be displaced in a crotch part of the wear. In an embodiment of the present disclosure, the wing portions 55 each are formed in a region from a front wing-base 55f to a back wing-base 55b in the longitudinal direction. This region is also a region to contact a crotch part (vaginal orifice) of a wearer when wearing the napkin 1.

The position in the longitudinal direction of the front wing-base 55f is defined as follows. That is, a distance in the width direction between outer edges on both ends in the width direction of the napkin 1 is set as a width W55, and when moving, along the outer edge of the wing portion 55, from the central position in the longitudinal direction of the wing anti-displacement portion 135 to the front side, a position in the longitudinal direction at which the width W55 is shifted from decreasing to increasing is defined as the front wing-base 55f. Further, a distance between the front wing-bases 55f provided on both sides in the width direction is defined as a front wing-base width W55f. In FIG. 1, the front wing-base width W55f is the shortest in a region on the front side with respect to the central position in the longitudinal direction of the wing anti-displacement portion 135.

Similarly, when moving, along the outer edge of the wing portion 55, from the central position in the longitudinal direction of the wing anti-displacement portion 135 to the back side, a position in the longitudinal direction at which the width W55 is shifted from decreasing to increasing is defined as the back wing-base 55b. Further, a distance between the back wing-bases 55b provided on both sides in the width direction is defined as a back wing-base width W55b. In FIG. 1, the back wing-base width W55b is the shortest in a region on the back side with respect to the central position in the longitudinal direction of the wing anti-displacement portion 135.

Further, when the napkin 1 is worn, the wing portions 55 each are folded along a reference folding line, which is a straight line in the longitudinal direction passing thorough the base, out of the front wing-base 55f and the back wing-base 55b, at which the width W55 (W55f or W55b) is shorter. In FIG. 1, since the width W55f at the front wing-bases 55f is shorter than the width W55b at the back wing-bases 55b (W55f<W55b), a straight line along the longitudinal direction passing through the front wing-base 55f serves as a reference folding line FLW of the wing portion 55.

The hip flap portions 56 are provided to extend outward in the width direction on the back side (back side) in the longitudinal direction in the napkin 1, and when the napkin 1 is worn, the hip flap portions 56 are in a state spread out in the width direction as illustrated in FIG. 2, to be attached to a buttock region of a wearer's underwear using the hip-flap anti-displacement portions 136. As a result, the wearer's buttock part is broadly covered with the hip flap portions 56. For example, even if the wearer is lying on his/her back and blood discharge and the like reach the buttock side, since the hip flap portions 56 cover the buttock part in a wide range, such blood discharge and the like are less likely to leak outside the napkin 1, so that the wearer's underwear can be prevented from being soiled.

Further, as illustrated in FIGS. 1 and 3, a plurality of compressed parts is formed on the skin-side surface of the napkin 1, so that the top sheet 20 (and the second sheet 22) and the absorbent body 10 are compressed together in the thickness direction to be integrally joined. In the top sheet 20 according to an embodiment of the present disclosure, linear compressed parts EL20 are formed as such compressed parts. The plurality of linear compressed parts EL20 is formed to run in lines along an outer peripheral edge portion of the absorbent body 10, thereby forming a substantially annular shape elongated in the longitudinal direction as a whole. Further, compressed parts other than the linear compressed parts EL20, as illustrated in FIG. 1, may be formed. For example, a plurality of dotted compressed parts may be provided on the front surface of the top sheet 20.

Further, in the napkin 1, the top sheet 20 (and the side sheets 25) and the back sheet 30 have the same planar shape. As illustrated in FIGS. 1 and 2, the outer peripheral portions of these sheets are at least partially joined by means of welding or the like, to form a the surround seal 70. However, in an embodiment of the present disclosure, the surround seal 70 is not an essential component, but the top sheet 20 and the back sheet 30 may be joined by other means.

### Compressed Region 200

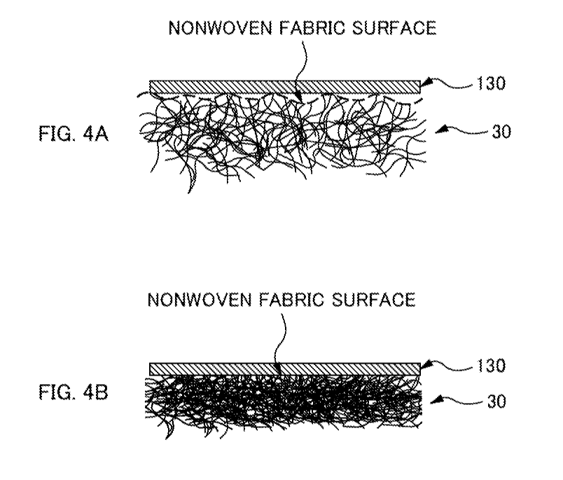

The compressed region 200 formed on the back sheet 30 of the napkin 1 will be described. FIGS. 4A and 4B are diagrams illustrating the states of a surface of a nonwoven fabric before and after being subjected to the compression process.

FIG. 4A schematically illustrates the state of the surface of a common nonwoven fabric not subjected to the compression process. As described above, the back sheet 30 according to an embodiment of the present disclosure is formed with a nonwoven fabric. Then, the anti-displacement adhesive portion 130 (main-body anti-displacement portion 131, wing anti-displacement portions 135, hip-flap anti-displacement portions 136) are famed on the non-skin side of the back sheet 30, while the leak-proof sheet adhesive portion 140 is formed on the skin side of the back sheet 30. That is, an adhesive is provided on the surfaces of the nonwoven fabric. In general, on the surface of a nonwoven fabric, a plurality of fibers is entangled in a complex manner to fault fine asperities, and the surface is rough microscopically. When an adhesive is provided on such a surface, the area (the area of contact) of the portion in which the adhesive contacts the surface of the nonwoven fabric results in being small, as illustrated in FIG. 4A, so that the adhesive (adhesive portion) does not sufficiently adhere to the surface of the nonwoven fabric, and is likely to be peeled off.

For example, in the napkin 1, if the anti-displacement adhesive portion 130 is likely to be peeled off, the so-called “adhesive residue”, which indicates that the adhesive is peeled off from the back sheet 30 and remains on underwear, may be caused, when the anti-displacement adhesive portion 130 is peeled off from the underwear, after use of the napkin 1. Further, in the napkin 1, if the leak-proof sheet adhesive portion 140 is likely to be peeled off, the leak-proof sheet adhesive portion 140 may be peeled off, thereby separating the leak-proof sheet 40 from the back sheet 30, so that the napkin 1 may be torn, when the used napkin 1 is removed from the underwear.

Thus, in an embodiment of the present disclosure, the compression process is performed onto a predetermined region in the back sheet 30 made of a nonwoven fabric, to form the compressed region 200, so that the adhesive is less likely to be peeled off in the compressed region 200. FIG. 4B is a diagram schematically illustrating the state of the surface of the nonwoven fabric subjected to the compression process. In FIG. 4B, fibers are pressed by the compression process, so that the fine asperities on the surface of the nonwoven fabric are reduced to bring about a smooth state on the surface. When an adhesive is provided on such a surface, the area (the area of contact) of the portion in which the adhesive contacts the surface of the nonwoven fabric results in being larger, so that the adhesion of an adhesive can be strengthened as compared with that in the case of FIG. 4A.

Note that the compression process is performed such that a predetermined region in the back sheet 30 is subjected to a known pressurizing process such as a calendaring process and an embossing process. Further, in the napkin 1, the density of the compressed region 200 formed by the compression process is not necessarily uniform. That is, a high density portion having a high density and a low density portion having a density lower than that of the high density portion may be formed in the compressed region 200. In the present specification, the entire region (including the high density portion and the low density portion) that is subjected to the compression process as such is defined as a compressed region, while a region not subjected to the compression process is defined as an uncompressed region. Note that, in the uncompressed region, the nonwoven fabric is in such a surface state as illustrated in FIG. 4A, and the surface and its texture are softer than those in the compressed region 200.

Subsequently, the surface characteristics of the compressed region 200 formed in the back sheet 30 will be specifically described. In an embodiment of the present disclosure, “wettability” is used as an index of smoothness of the surface of the compressed region 200, where the lower the wettability is, the higher the water repellency is, and the smoother the surface of the compressed region 200 is. The degree of wettability in the compressed region 200 can be measured by a wettability test.

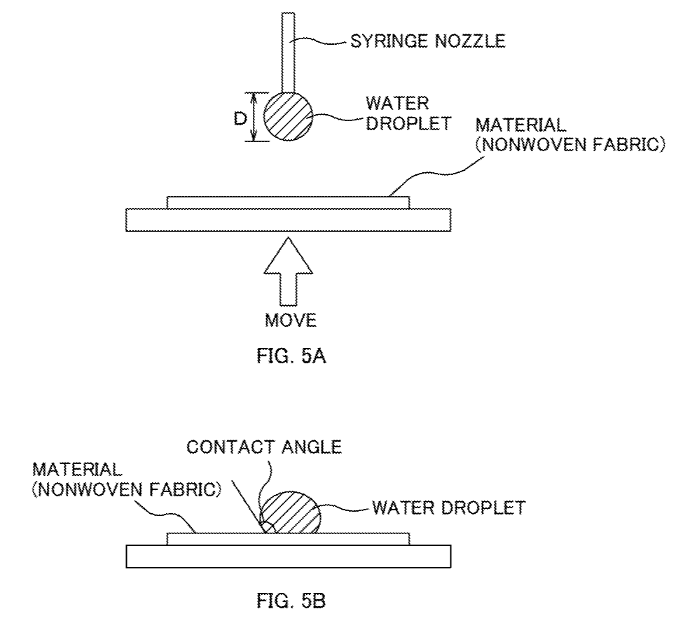

FIGS. 5A and 5B are diagrams illustrating a wettability test method. In the wettability test, first, a droplet of a predetermined size (e.g., diameter D) is formed (for example, a given amount of water is discharged from a nozzle of a syringe to form a droplet) as in FIG. 5A. Thereafter, a material (nonwoven fabric) provided on a stage disposed on the lower side is moved vertically upward toward the droplet, so that the droplets contact the material. Then, as in FIG. 5B, the contact angle of the droplet is measured in a state in which the droplet is in contact with the material on the material. In the wettability test, a plurality of measurements are performed onto each of a plurality of types of materials subjected to the compression process under conditions, such as a pressure at the time of the compression process (0, 10, 20, 30 kg/cm2), a heating temperature at the time of the compression process, and a transport speed, etc., being changed, to obtain averages of the measurement values of the contact angles.

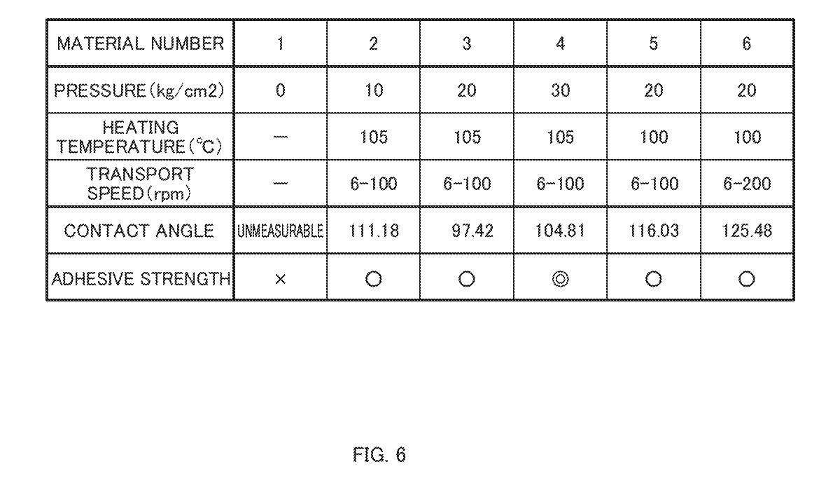

FIG. 6 is a table representing results of the wettability test. First, in the case of the material number 1 which is a nonwoven fabric not subjected to the compression process (when a pressure is 0 kg/cm2), since water penetrates through the surface of the nonwoven fabric, the contact angle was unmeasurable. Under the conditions other than that, contact angles were measured and degrees from 97.42 to 125.48 were obtained. Further, the same types of materials as those in the wettability test were used to conduct a test in which an adhesive is peeled off after being applied to a surface, so as to demonstrate the adhesive strength. As a result, the adhesive has a sufficient adhesive strength under all the conditions of the contact angles from 97.42 to 125.48 degrees, thereby being able to confirm that the adhesive is less likely to be peeled off.

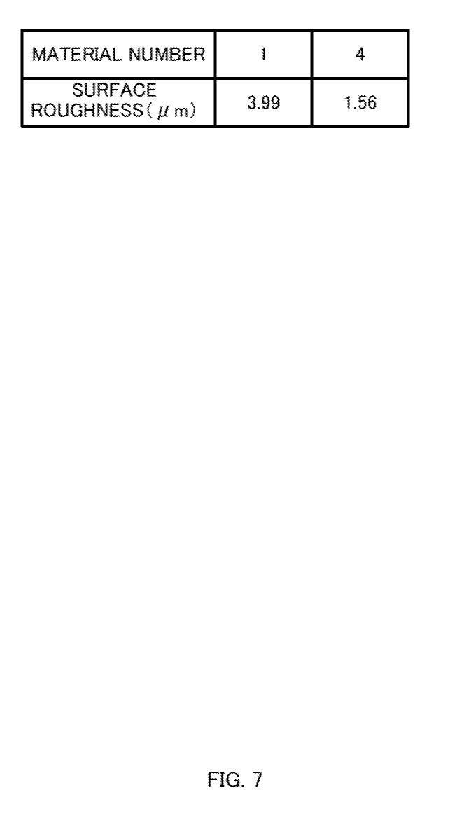

Further, for the specimen of the material numbers 1 and 4, surface roughness was measured using an automatic surface tester “KES-FB4 auto-A” manufactured by Kato Tech Co., Ltd. The tester performs a friction test on a surface of a material to be tested using a predetermined friction unit (sensor), and is able to measure frictional coefficients of a material surface, fluctuations of frictional coefficients, surface roughness, and the like. In an embodiment of the present disclosure, a contactor of 5 mm×5 mm is used to perform a plurality of surface tests (three times for each of the specimen) under the condition of a rough static load of 10 g, to measure surface roughnesses and record average values thereof. FIG. 7 is a table representing the results of the surface test. As illustrated in FIG. 7, in the nonwoven fabric of the material number 1 not subjected to the compression process, the average surface roughness was 3.99 μm. On the other hand, in the nonwoven fabric of the material number 4 to which the compression process is strongly performed, the average surface roughness is 1.56 μm. From this data, it is numerically clear that the smoothness is higher on the surface of the nonwoven fabric subjected to the compression process, than on the surface of the nonwoven fabric not subjected to the compression process.

Accordingly, if the contact angle of water on the compressed region 200 is in a range from 100 to 120 degrees as an index of wettability representing smoothness of the surface of the compressed region 200, the surface of the compressed region 200 is considered to have a sufficient smoothness. Accordingly, the adhesion of the anti-displacement adhesive portion 130 and the leak-proof sheet adhesive portion 140 in the compressed region 200 are strengthened, and the adhesive can be less likely to be peeled off from the back sheet 30.

### Relationship Between Anti-Displacement Adhesive Portion 130 and Compressed Region 200

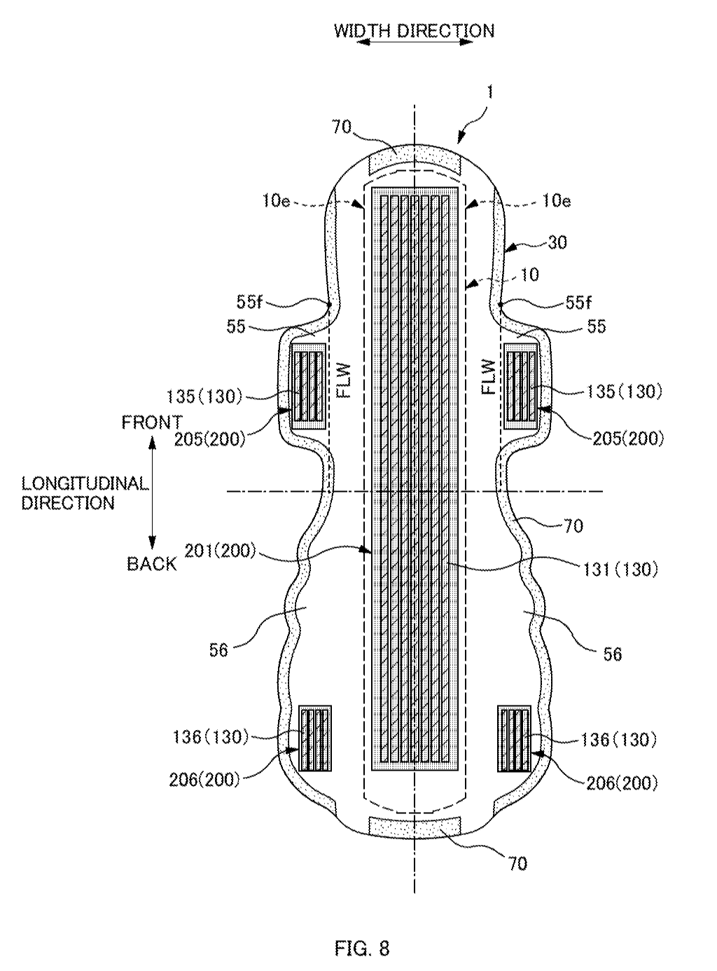

FIG. 8 is a plan view illustrating a relationship between the anti-displacement adhesive portion 130 and the compressed region 200. In the napkin 1, a main-body compressed region 201, wing compressed regions 205, and hip-flap compressed regions 206 are formed as the compressed region 200. As indicated by a hatching portion in FIG. 8, the main-body compressed region 201 is a rectangular region disposed along the longitudinal direction while having a predetermined width in the central portion in the width direction. In the main-body compressed region 201, the main-body anti-displacement portion 131 is formed. That is, the main-body anti-displacement portion 131 is formed so as to overlap the main-body compressed region 201. As described above, the main-body compressed region 201 (compressed region 200) has higher wettability and the surface of the nonwoven fabric is formed smooth. This increases the contact area between the surface of the nonwoven fabric and the adhesive, thereby increasing its adhesion. Thus, the adhesive forming the main-body anti-displacement portion 131 firmly adheres to the surface of the back sheet 30 (main-body compressed region 201), thereby being made less likely to be peeled off. Accordingly, when removing the used napkin 1 from underwear, the “adhesive residue”, which indicates that the adhesive forming the main-body anti-displacement portion 131 remains on underwear, can be effectively restrained.

The wing compressed regions 205 are rectangular regions respectively formed in the wing portions 55. In the wing compressed regions 205, the wing anti-displacement portions 135 are formed, respectively. That is, each wing anti-displacement portion 135 formed so as to overlap each wing compressed region 205. Similarly, the hip-flap compressed regions 206 are rectangular regions respectively formed in the hip flap portions 56. That is, in the hip-flap compressed regions 206, the hip-flap anti-displacement portions 136 are formed, respectively. That is, each hip-flap anti-displacement portion 136 is formed so as to overlap each hip-flap compressed region 206. Thus, the adhesives forming the wing anti-displacement portion 135 and the hip-flap anti-displacement portion 136 firmly adhere to the surface of the back sheet 30, thereby being less likely to be peeled off.

Note that the shapes of the compressed region 200 and the anti-displacement adhesive portion 130 are not limited to the shapes illustrated in FIG. 8. For example, even if the compressed region 200 and the anti-displacement adhesive portion 130 overlap each other at only a part of the regions, the adhesion of the adhesive can be strengthened in this overlapping region. Thus, the anti-displacement adhesive portion 130 can be less likely to be peeled off from the back sheet 30.

Further, a portion in which the compressed region 200 is not formed in the back sheet 30 results in an uncompressed region not subjected to the compression process. That is, the back sheet 30 of the napkin 1 includes the compressed region 200 and the uncompressed region. In this uncompressed region, the surface of the nonwoven fabric is maintained softer than that in the compressed region 200. Thus, the texture is not likely to deteriorate even when contacting the skin of a wearer wearing the napkin 1. That is, the back sheet 30 of the napkin 1 includes the uncompressed region to ensure preferable texture.

In FIG. 8, a region between the main-body compressed region 201 and each of the wing compressed regions 205 in the width direction is the uncompressed region. That is, the napkin 1 includes the uncompressed region between the main-body anti-displacement portion 131 and each of the wing anti-displacement portions 135. In specific, a region in which the reference folding line FLW of the wing portion 55 is provided is the uncompressed region. When the napkin 1 is worn, the wing portion 55 is folded along the reference folding line FLW. Thus, if the reference folding line FLW is formed in the uncompressed region that is softer than the compressed region 200, the wing portion 55 can be easily folded, so that the napkin 1 can be easily worn. Further, the position of the reference folding line FLW is a portion more likely to directly contact both legs of a wearer when wearing the napkin 1. Thus, if this portion is in the uncompressed region, the texture is soft, so that the wearer is less likely to feel discomfort.

Further, as illustrated in FIG. 8, in the napkin 1, the outer edges (edges) on both sides in the width direction of the absorbent body 10 are the uncompressed regions. The edges on both sides in the width direction of the absorbent body 10 are portions to be caught by the inguinal regions of a wearer when wearing the napkin 1. That is, the edges are portions to be strongly pressed against the inguinal regions of the wearer. Thus, if such outer edges 10e on both sides in the width direction of the absorbent body 10 overlap the compressed region 200, the nonwoven fabric whose surface is hardened by the compression process is pressed against the wearer's inguinal regions, which is likely to cause discomfort. In contrast, in the napkin 1, the outer edges 10e on both sides in the width direction of the absorbent body 10 overlap the soft uncompressed regions. This enables preferable touch at the wearer's inguinal regions.

Further, the compressed region 200 is formed so as not to overlap the outer peripheral edge (edge) of the napkin 1. That is, the outer peripheral edge of the napkin 1 is the uncompressed region. In the longitudinal direction, particularly, the main-body compressed region 201 is disposed so as not to overlap the outer peripheral edge of the napkin 1. If the surface of the nonwoven fabric is hardened at both edges in the longitudinal direction of the napkin 1, the edges are likely to be caught by the front portion and back portion (buttock portion) of a wearer when wearing the napkin 1, which may cause discomfort to the wearer. Thus, by forming the edges into the uncompressed regions, flexibility is ensured, so as to cause less discomfort to the wearer. The wing compressed regions 205 and the hip-flap compressed regions 206 do not overlap, in the longitudinal direction, with the outer peripheral edge of the napkin 1. Similarly, in the lateral direction, the wing compressed regions 205 and the hip-flap compressed regions 206 are disposed so as not to overlap the outer peripheral edge of the napkin 1. The edges of the wing portions 55 and the hip flap portions 56 are the portions to contact both legs and buttock part of a wearer when wearing the napkin 1. Thus, the edges are formed into the uncompressed regions, to ensure flexibility of the surface of the nonwoven fabric, so as not to cause discomfort to the wearer.

Further, in the napkin 1, the compressed region 200 is formed so as not to overlap the surround seal 70 of the napkin 1. The surround seal 70 is a region having a predetermined width along the outer peripheral edge of the napkin 1. In this region, the top sheet 20 (side sheets 25) and the back sheet 30 are joined to each other. Accordingly, in this region, if the surface of the back sheet 30 (i.e., the surface to contact a wearer's skin) is hardened, the wearer is likely to feel the hardness of the surround seal 70, so that awkwardness when wearing the napkin 1 may be increased. Thus, the surround seal 70 is formed into the uncompressed region, to ensure flexibility of the surface of the nonwoven fabric, so as to cause less awkwardness when wearing the napkin 1. Further, with such an arrangement in which the compressed region 200 does not overlap the surround seal 70, the compressed region 200 can be less likely to overlap the outer peripheral edge (edge) of the napkin 1.

### Relationship Between Compressed Region 200, and Leak-Proof Sheet 40 and Leak-Proof Sheet Adhesive Portion 140

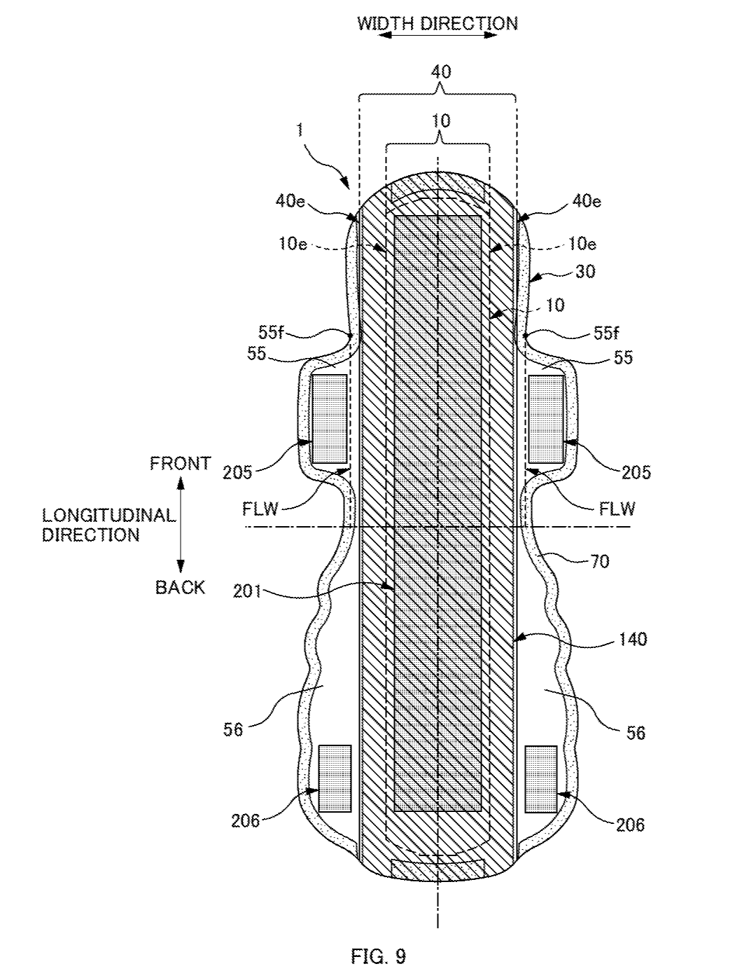

FIG. 9 is a plan view illustrating a relationship between the compressed region 200 (main-body compressed region 201), and the leak-proof sheet 40 and leak-proof sheet adhesive portion 140. As illustrated in FIG. 9, the leak-proof sheet adhesive portion 140 (indicated by a shaded area in FIG. 9) includes a portion overlapping the main-body compressed region 201, as well as is formed across substantially all the region of the leak-proof sheet 40. As described above, in the main-body compressed region 201, the surface of the nonwoven fabric is smooth, and thus the adhesive firmly adheres in a region in which the main-body compressed region 201 overlaps the leak-proof sheet adhesive portion 140, so that the adhesive is less likely to be removed. This makes it easier to restrain the used napkin 1 from being torn due to peeling of the leak-proof sheet 40 (leak-proof sheet adhesive portion 140) from the back sheet 30 when the used napkin 1 is removed from underwear.

The leak-proof sheet adhesive portion 140 is formed so that an adhesive such as a hot-melt adhesive (HMA) is applied to the back sheet 30 using an omega patter, a spiral pattern, a coater with 1-mm pitches or the like. That is, in the leak-proof sheet adhesive portion 140, the adhesive is intermittently provided to the material (back sheet 30) without so-called “being applied solidly”. As described above, the leak-proof sheet 40 is formed of a breathable film. However, if the adhesive is solidly applied in the leak-proof sheet adhesive portion 140, breathability deteriorates, which is likely to cause stuffiness while the napkin 1 is worn. Thus, the adhesive is intermittently provided, so as to ensure the minimum necessary adhesion without losing breathability.

Further, in an embodiment of the present disclosure, the back sheet 30 is subjected to the compression process to form the compressed region 200, so as to restrain the back sheet 30 itself from being torn. That is, the tensile strength of the nonwoven fabric in the compressed region 200 is made equal to or greater than a predetermined strength, so as to restrain the nonwoven fabric from being torn. The tensile strength of the nonwoven fabric can be measured by a tensile strength test as follows. First, a plurality of specimen (e.g., 10 pieces) of a material to be measured (back sheet 30 subjected to the compression process), obtained by cutting the material into pieces of substantially 10 mm×10 mm, is prepared, and a tensile test is conducted on each of the specimen using a commercially available tensile tester. In specific, the specimen are extended until they reach the predetermined rate of elongation (e.g., 3%, 5%, 10%, etc.), and the tensile strengths are measured at that time to calculate the average value. The materials to be measured are a nonwoven fabric that is not subjected to the compression process and three types of nonwoven fabrics that are subjected to the compression process while the pressure during compression is being changed in a stepwise manner (e.g., 10 (weak), 20 (medium), 30 (strong) kg/cm2).

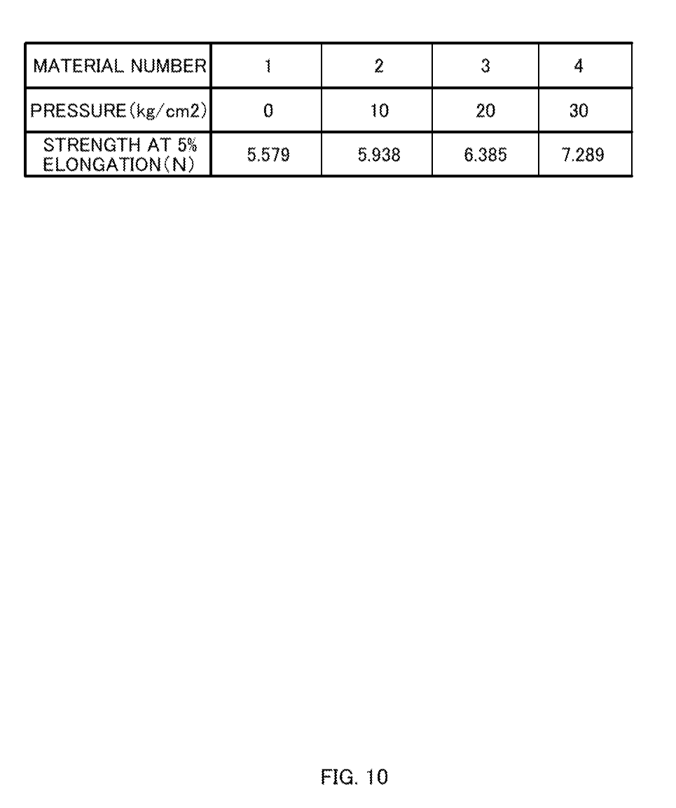

FIG. 10 is a table representing the results of the tensile test. FIG. 10 shows the measurement results of the tensile test when the specimen are extended at 5%. Note that since the maximum rate of elongation of the nonwoven fabric in removing the napkin 1 from underwear is 5% or lower, the tensile strength with an elongation of 5% can be the maximum strength required for the back sheet 30. As a result of measurement, the strength of the nonwoven fabric (material number 1) that was not subjected to the compression process was 5.579 N with an elongation of 5%. On the other hand, the strengths of the nonwoven fabrics that were subjected to the compression process while the pressure during the compression process was being changed in a range of 10 to 30 (kg/cm2) were larger than 5.579 N with an elongation of 5%. That is, it was confirmed that the formation of the compressed region 200 increases the strength of the back sheet 30 itself. This is because the compression process increases the density of the fibers constituting the nonwoven fabric, and also increases the fusion points among fibers, thereby strengthening the bonding force among fibers. Accordingly, in the back sheet 30 according to an embodiment of the present disclosure, the compressed region 200 is formed so as to have a tensile stress equal to or greater than 6 N when the nonwoven fabric is elongated at 5%. This makes it easier to restrain the back sheet 30 itself from being torn when the napkin 1 is removed from underwear.

Further, in the napkin 1 according to an embodiment of the present disclosure, the length in the width direction of the leak-proof sheet 40 is longer than the length in the width direction of the absorbent body 10. In other words, both ends in the width direction of the leak-proof sheet 40 are positioned outside both the ends 10e in the width direction of the absorbent body 10. The width of the leak-proof sheet 40 is made larger than that of the absorbent body 10, so that the moisture of blood discharge and the like absorbed by the absorbent body 10 is likely to be suppressed from moving to the back sheet 30 on the non-skin side.

Further, both the ends 40e in the width direction of the leak-proof sheet 40 are respectively positioned inside, in the width direction, the reference folding lines FLW of the wing portions 55. This suppresses the leak-proof sheet 40 from being folded together with the wing portions 55 when the wing portions 55 are respectively folded at the reference folding lines FLW to fix the napkin 1 to underwear. The leak-proof sheet 40 is constituted by a material harder than the nonwoven fabric, and thus if the leak-proof sheet 40 is disposed to overlap the positions of the reference folding lines FLW of the wing portion 55, this may cause a wearer to feel hard texture when the reference folding lines FLW contact the wearer's legs, which may cause awkwardness to the wearer. In contrast, in an embodiment of the present disclosure, the leak-proof sheet 40 is disposed inside, in the width direction, the reference folding lines FLW, which is less likely to cause awkwardness to the wearer.

Further, in the napkin 1, the main-body compressed region 201 is provided so as to become narrower than the leak-proof sheet 40, as well as become wider than the main-body anti-displacement portion 131 (see FIGS. 8 and 9). Since the non-skin-side face of the back sheet 30 may directly contact a wearer's skin, it is preferable that a region where the main-body compressed region 201 is formed is made as small as possible, so that the texture of the surface of the nonwoven fabric (back sheet 30) in this region is maintained soft. In an embodiment of the present disclosure, the main-body compressed region 201 is made narrower than the leak-proof sheet 40, so that the non-skin-side surface of the back sheet 30 is made broader than the main-body anti-displacement portion 131 while a soft region of the non-skin-side surface being maintained as broad as possible. This restrains the adhesive of the main-body anti-displacement portion 131 from being peeled off from the back sheet 30. Further, with this structure, the leak-proof sheet adhesive portion 140 and the main-body anti-displacement portion 131 are disposed to overlap each other in the thickness direction while sandwiching the main-body compressed region 201, so that their respective adhesives are less likely to be peeled off. Accordingly, in a region in which the greatest force is exerted in removing the used napkin 1 from underwear, it is possible to effectively restrain the napkin 1 from being torn due to peeling of the leak-proof sheet adhesive portion 140, while restraining the generation of an adhesive residue of the main-body anti-displacement portion 131.

Furthermore, the main-body compressed region 201 is provided so as to become narrower than the absorbent body 10. Accordingly, when the napkin 1 is worn, the absorbent body 10 is always disposed between the wearer's skin and the main-body compressed region 201. In the main-body compressed region 201, the surface of the nonwoven fabric is hardened by the compression process. However, the absorbent body 10 serves as a cushion so that a wearer is not likely to feel the hardness of the main-body compressed region 201, which causes less awkwardness to the wearer.

### Modified Example of Compressed Region 200

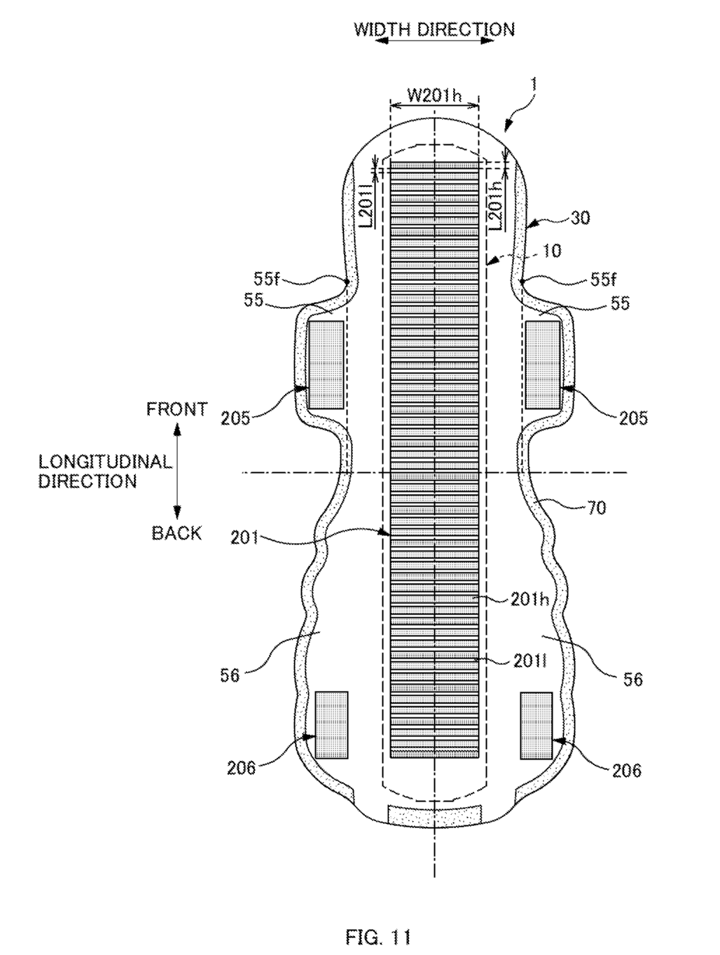

As a modified example of the compressed region 200, an example will be described in which a high density portion having a high density, and a low density portion having a density lower than that of the high density portion, are formed in the main-body compressed region 201. FIG. 11 is a diagram illustrating a modified example of the main-body compressed region 201. In the present modified example, the main-body compressed region 201 includes high density portions 201h (shaded portions in FIG. 11) and low density portions 201l. The high density portions 201h each are a rectangular region having a length W201h in the width direction and a length L201h in the longitudinal direction, and the high density portions 201h are disposed intermittently at predetermined intervals in the longitudinal direction. The low density portions 201l each are disposed between every two, of the high density portions 201h, adjacent to each other. That is, the main-body compressed region 201 is formed such that the high density portions 201h and the low density portions 201l are arranged alternately in the longitudinal direction.

The low density portion 201l is likely to maintain softness of the surface of the nonwoven fabric. Thus, in the main-body compressed region 201 of the modified example, the provision of the low density portions 201l allows the main-body compressed region 201 to be more likely to maintain softness, as compared with the case where the high density portions 201h are formed in the entire region of the high density portions 201h. Also, in the main-body anti-displacement portion 131 formed to overlap such a main-body compressed region 201, its softness and texture is guaranteed to some extent.

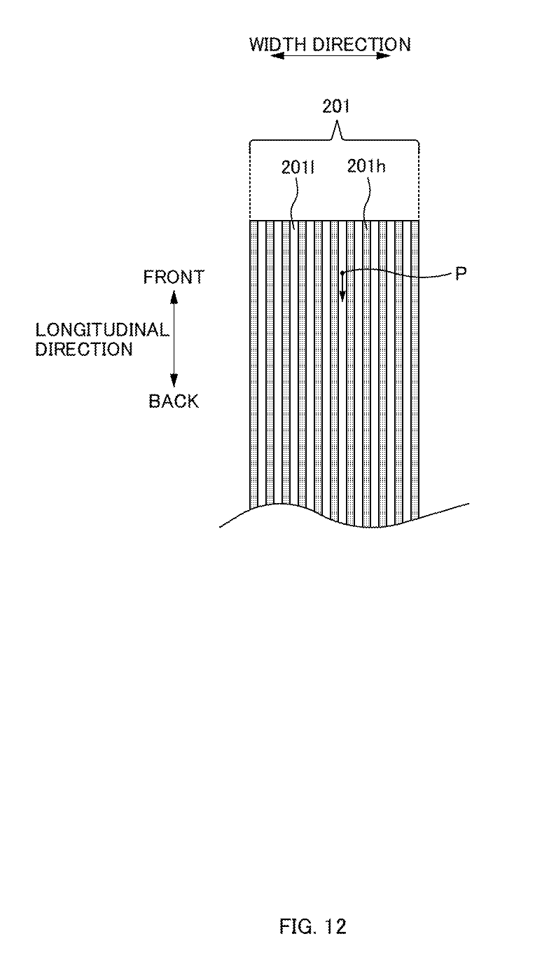

Further, when the main-body compressed region 201 is provided with the high density portions 201h and the low density portions 201l, the high density portions 201h are provided intermittently in the longitudinal direction, so that the adhesive of the main-body anti-displacement portion 131 can be made less likely to be peeled off when the used napkin 1 is removed from underwear. FIG. 12 is a diagram illustrating the case where the high density portions 201h and the low density portions 201l are continuously arranged in the longitudinal direction in the compressed region 200. When removing the napkin 1, the napkin 1 is pulled along the longitudinal direction. In this case, if the low density portion 201l is continuously formed in the longitudinal direction as illustrated in FIG. 12, and if the adhesive is peeled anywhere in the low density portion 201l, the adhesive may be continuously peeled along the low density portion 201l extending in the longitudinal direction. For example, if the adhesive is peeled at a point P in FIG. 12 when removing the napkin 1, peeling of the adhesive is likely to propagate in the longitudinal direction from the point P serving as a starting point. In contrast, as long as the high density portions 201h and the low density portions 201l are formed in a striped manner intermittent in the longitudinal direction, as in FIG. 11, even if the adhesive is peeled in any region in the low density portion 201l, propagation of such peeling of the adhesive in the longitudinal direction can be stopped at the high density portion 201h that is adjacent to the low density portion 201l in the longitudinal direction. Accordingly, by disposing the high density portions 201h (and the low density portions 201l) intermittently in a direction of removing the main-body compressed region 201 (longitudinal direction), the adhesive is restrained from being peeled continuously, so that the adhesive can be made less likely to be peeled off.

Note that, in the napkin 1, the length L201h in the longitudinal direction of the high density portion 201h is formed longer than a length L201l in the longitudinal direction of the low density portion 201l (L201h>L201l). Accordingly, in the main-body compressed region 201, the area of the high density portions 201h becomes larger than the area of the low density portions 201l. Thus, a smooth portion is larger in the surface of the back sheet 30 (nonwoven fabric), so that the adhesive is further less likely to be peeled.

Further, in an embodiment of the present disclosure, an interval between every two, of the high density portions 201h, adjacent in the longitudinal direction (i.e., the length L201l in the longitudinal direction of the low density portion 201l) is set to 3 mm or smaller. With a region between the high density portions 201h being set to a predetermined size (3 mm) or smaller, the region of the low density portion 201l is limited, so that the adhesive is restrained from being peeled off at the low density portion. Further, in an embodiment of the present disclosure, the main-body anti-displacement portion 131 is formed as each region having at least a width equal to or greater than 3 mm, to ensure the minimum necessary adhesion. Thus, when a space between every two, of the high density portions 201h, adjacent to each other in the longitudinal direction is equal to or smaller than 3 mm, the main-body anti-displacement portion 131 at least partially overlaps the high density portions 201h. This can further restrain the adhesive from being peeled.

### Other Embodiments

The above embodiments of the present disclosure are simply to facilitate understanding of the present disclosure and are not in anyway to be construed as limiting the present disclosure. The present disclosure may variously be changed or altered without departing from its gist and encompass equivalents thereof.

In an embodiment described above, the absorbent article (sanitary napkin 1) including the side flaps 50 has been described, however, the side flaps 50 may not be necessarily provided. For example, the absorbent article may be an absorbent article without the wing portions 55 or the hip flap portions 56.

### REFERENCE SIGNS LIST

1 sanitary napkin (napkin),

10 absorbent body, 10e both ends in the width direction,

20 top sheet, 22 second sheet, 25 side sheet, 30 back sheet,

40 leak-proof sheet, 40e both ends in the width direction,

50 side flap, 51 reinforcement sheet,

55 wing portion, 51f front wing-base, 51b back wing-base,

56 hip flap portion,

70 surround seal,

130 anti-displacement adhesive portion,

131 main-body anti-displacement portion,

135 wing anti-displacement portion, 136 hip-flap anti-displacement portion,

140 leak-proof sheet adhesive portion,

200 compressed region,

201 main-body compressed region, 201h high density portion, 201l low density portion,

205 wing portion compressed region, 206 hip flap portion compressed region,

EL20 linear compressed part,

FLW reference folding line

1-11. (canceled)

12. An absorbent article having a longitudinal direction, a width direction, and a thickness direction that intersect with one another, the absorbent article comprising:
an absorbent body that absorbs liquid;
a back sheet disposed on a wearer's non-skin side of the absorbent body, the back sheet made of a nonwoven fabric; and
an anti-displacement adhesive portion formed with an adhesive, the adhesive provided on a non-skin side of the back sheet,
the back sheet including a compressed region that is compressed in the thickness direction,
the back sheet including a portion where the anti-displacement adhesive portion overlaps the compressed region,
the compressed region including high density portions having a high density and low density portions having a density lower than the density of the high density portions,
the compressed region having the high density portions disposed intermittently in the longitudinal direction, the high density portions having a predetermined length in the width direction,
the compressed region having the low density portions each disposed between every two, of the high density portions, adjacent to each other in the longitudinal direction, the low density portions having a predetermined length in the width direction,
the high density portions each having a length, in the longitudinal direction, longer than a length in the longitudinal direction of each of the low density portions.

13. An absorbent article according to claim 12, wherein
the back sheet includes an uncompressed region in a region other than a region where the compressed region is formed, and
the uncompressed region overlaps a region where the anti-displacement adhesive portion is not disposed, in the back sheet.

14. An absorbent article according to claim 12, wherein
a contact angle of water in the compressed region is equal to or greater than 100 degrees and equal to or smaller than 120 degrees.

15. An absorbent article having a longitudinal direction, a width direction, and a thickness direction that intersect with one another, the absorbent article comprising:
an absorbent body that absorbs liquid;
a back sheet disposed on a wearer's non-skin side of the absorbent body, the back sheet made of a nonwoven fabric; and
an anti-displacement adhesive portion formed with an adhesive, the adhesive provided on a non-skin side of the back sheet,
the back sheet including a compressed region that is compressed in the thickness direction,
the back sheet including an uncompressed region in a region other than a region where the compressed region is formed, and
the back sheet including wing portions respectively extending outward from both side portions in the width direction of the absorbent body,
the compressed region including
a main-body compressed region formed along the longitudinal direction in a central portion in the width direction, and
wing compressed regions formed in the wing portions, respectively,
the anti-displacement adhesive portion including
a main-body anti-displacement portion formed along the longitudinal direction in a central portion in the width direction, and
wing anti-displacement portions formed on a non-skin side of the wing portions, respectively,
the absorbent article including a portion where the main-body anti-displacement portion overlaps the main-body compressed region,
the absorbent article including a portion where the wing anti-displacement portions overlap the wing compressed regions, respectively,
the uncompressed region is provided in a region between the main-body anti-displacement portion and each of the wing anti-displacement portions in the width direction.

16. An absorbent article according to claim 12, wherein
the back sheet includes an uncompressed region in a region other than a region where the compressed region is formed, and
both ends in the width direction of the absorbent body overlap the uncompressed region.

17. An absorbent article according to claim 12, wherein
the compressed region does not overlap an outer peripheral edge of the absorbent article.

18. An absorbent article according to claim 17, further comprising;
a top sheet disposed on a wearer's skin side of the absorbent body; and
a surround seal portion joining the top sheet and the back sheet at an outer peripheral portion of the absorbent article, wherein
the compressed region does not overlap the surround seal portion.

19. A method for manufacturing an absorbent article, comprising:
forming a compressed region by performing a compression process, in the thickness direction, onto a predetermined region of a back sheet made of a nonwoven fabric;
forming an anti-displacement adhesive portion by providing an adhesive onto a non-skin side of the back sheet, the anti-displacement adhesive portion including a portion overlapping the compressed region; and
joining an absorbent body that absorbs liquid to a skin side of the back sheet,
the compressed region including high density portions having a high density and low density portions having a density lower than the density of the high density portions,
the compressed region having the high density portions disposed intermittently in the longitudinal direction, the high density portions having a predetermined length in the width direction,
the compressed region having the low density portions each disposed between every two, of the high density portions, adjacent to each other in the longitudinal direction, the low density portions having a predetermined length in the width direction,
the high density portions each having a length in the longitudinal direction longer than a length in the longitudinal direction of each of the low density portions.

20. A method for manufacturing an absorbent article, comprising:
forming a compressed region by performing a compression process, in the thickness direction, onto a predetermined region of a back sheet made of a nonwoven fabric;
forming an anti-displacement adhesive portion by providing an adhesive onto a non-skin side of the back sheet; and
joining an absorbent body that absorbs liquid to a skin side of the back sheet,
the absorbent article having a longitudinal direction, a width direction, and a thickness direction that intersect with one another,
the back sheet including an uncompressed region in a region other than a region where the compressed region is formed, and
the back sheet including wing portions respectively extending outward from both side portions in the width direction of the absorbent body,
the compressed region including
a main-body compressed region formed along the longitudinal direction in a central portion in the width direction, and
wing compressed regions formed in the wing portions, respectively,
the anti-displacement adhesive portion including
a main-body anti-displacement portion formed along the longitudinal direction in a central portion in the width direction, and
wing anti-displacement portions formed on a non-skin side of the wing portions, respectively,
the absorbent article including a portion where the main-body anti-displacement portion overlaps the main-body compressed region,
the absorbent article including a portion where the wing anti-displacement portions overlap the wing compressed regions, respectively,
the uncompressed region is provided in a region between the main-body anti-displacement portion and each of the wing anti-displacement portions in the width direction.
